(19) United States
(12) Patent Application Publication
(10) Pub. No.: US 2009/0312429 A1
(43) Pub. Date: Dec. 17, 2009

(54) Administration of nepafenac or derivatives thereof for treating dermatological/keratinization disorders

(75) Inventors: Irina Safonova, Nice (FR); Cecile Cousin, Mouans-Sartoux (FR); Leila Zarif, Peymeinade (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH&DEVELOPMENT, Biot (FR)

(21) Appl. No.: 12/457,754

(22) Filed: Jun. 19, 2009

(30) Foreign Application Priority Data
Dec. 19, 2006 (FR) 0655655
Dec. 19, 2007 (FR) PCT/FR2007/052559

(51) Int. Cl. A61K 31/165; C07C 237/14

(52) U.S. Cl. 514/619; 564/163

(57) ABSTRACT

Nepafenac or derivatives thereof are useful for the treatment of dermatological conditions related to a keratinization disorder that may have an inflammatory immunoallergic component, for example rosacea, acne, psoriasis or atopic dermatitis.

### CROSS-REFERENCE TO PRIORITY/PCT APPLICATIONS

This application claims priority under 35 U.S.C. § 119 of FR 06155655, filed Dec. 19, 2006, and is a continuation/national phase of PCT/FR 2007/052559, filed Dec. 19, 2007 and designating the United States (published in the French language on Jul. 17, 2008 as WO 2008/084171 A2; the title and abstract were also published in English), each hereby expressly incorporated by reference in its entirety and each assigned to the assignee hereof.

### BACKGROUND OF THE INVENTION

1. Technical Field of the Invention

The present invention relates to the administration of at least one compound of formula (I) or derivatives thereof, preferably nepafenac, formulated into pharmaceutical compositions for the treatment of dermatological conditions related to a keratinization disorder that may have an inflammatory immunoallergic component, in particular rosacea, acne, psoriasis or atopic dermatitis (eczema).

2. Description of Background and/or Related and/or Prior Art

Rosacea is a common chronic and progressive inflammatory dermatosis associated with vascular instability. It mainly affects the central part of the face and is characterized by redness of the face or hot flushes, facial erythema, papules, pustules and telangiectasia. In serious cases, particularly in men, facial elephantiasis may develop, most commonly in the form of swelling of the soft tissue of the nose, producing a bulbous swelling known as rhinophyma.

Rosacea generally occurs from the ages of 25 to 70, and is much more common in people of fair complexion. It more particularly affects women, although this condition is generally more severe in men. Rosacea is chronic and lasts for years with periods of exacerbation and of remission.

The pathogenesis of rosacea is poorly understood. Many factors may be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even an infection with Helicobacter pilori.

The minor forms of rosacea can be treated with topical treatments, for example metronidazole, azelaic acid, benzoyl peroxide or retinoic acid. As regards the more severe forms of the condition, they respond well to general antibiotic therapy with cyclins. However, these treatments have unpleasant side effects for the patient, such as irritation or intolerance phenomena.

Furthermore, on account of the multifactor aspect of rosacea, there are a very large number of treatments for this condition, but need continues for an effective treatment that is without risk to the patient.

The acne is a common multifactor pathology which affects skin rich in sebaceous glands (face, scapular area arms and intertriginous areas). It is the most commonly occurring form of dermatosis. The following five pathogenic factors play a determining role in the formation of acne:

1. genetic predisposition;

2. overproduction of sebum (seborrhea);

3. androgens;

4. follicular keratinization disorders (comedogenesis); and

5. bacterial colonization and inflammatory factors.

There are several forms of acne, the common factor of all of them being attack of the pilosebaceous follicles. Mention may in particular be made of acne conglobata, acne keloid of the nape of the neck, acne medicamentosa, recurrent miliary acne, acne necrotica, acne neonatorum, premenstrual acne, occupational acne, acne rosacea, senile acne, solar acne and acne vulgaris.

Acne vulgaris, also known as polymorphous juvenile acne, is the most common. It comprises four stages:

Stage 1 corresponds to comedonal acne, characterized by a large number of open and/or closed comedones and of microcysts.

Stage 2, or papulopustular acne, is of mild to moderate seriousness. It is characterized by the presence of open and/or closed comedones and of microcysts, but also of red papules and of pustules. It mainly affects the face and leaves few scars.

Stage 3, or papulocomedonal acne, is more serious and extends to the back, the thorax and the shoulders. It is accompanied by a larger number of scars.

Stage 4, or nodulocystic acne, is accompanied by numerous scars. It exhibits nodules and also has large painful purplish pustules.

The various forms of acne described above can be treated with active agents, such as anti-seborrheics and anti-infectives, for example benzoyl peroxide (in particular, the product Eclaran® marketed by Pierre Fabre), with retinoids, such as tretinoin (in particular, the product Retacnyl® marketed by Galderma), or isotretinoin (the product Roaccutane® marketed by Laboratoires Roche).

However, many side effects are induced by these treatments (in particular, dryness of the skin and of the mucous membranes, pain and swelling of the lips, desquamation of the skin, itching). There exists therefore a need for an effective treatment which does not pose any risk to the patient.

Similarly, atopic dermitis, or atopic dermatitis, or alternatively atopic eczema, is a pruriginous, chronic erythematovesicular-erythematosquamous inflammatory dermatosis which develops through exacerbations, and which essentially affects infants. The acute phase is characterized chronologically by:

an erythematous phase,

then vesicles with the skin having a crumbly appearance,

weeping and scabs, then

desquamation.

All these phases rapidly act in concert over time and the atopic dermatitis becomes chronic, with dry, erythematosquamous skin, cracks and lichenification of the lesions.

The principal constant criterion for diagnosis is pruritis. However, the following criteria can also be used to identify the pathology: dermatological history with the folds, the anterior face of the ankles or the neck being affected; history of xerosis (dryness of the skin); personal history of asthma or rhinitis; dermatosis of the folds or eczema of the cheeks, of the forehead and of the external face of the limbs in children less than 4 years old; beginning before the age of 2: ichthyosis and/or keratosis pilaris and/or palmar hyperlinearity, a tendency towards skin infections, nipple eczema, cheilitis, recurrent conjunctivitis, Denny Morgan fold, keratoconus, anterior subcapsular cataract, periorbital pigmentation, pityriasis alba (dry white patches), irritation of the anterior folds of the neck, perspiration-induced pruritus, intolerance to wool and to lipid solvents, white dermographism or delayed appearance of a white line in response to scratching, worsening of lesions under the influence of environmental and emotional factors.

Atopic dermatitis is probably worsened by pollution, but also, paradoxically, good hygiene with the use of detergents which detrimentally alter the skin barrier. In certain cases, worsening may occur due to dietary factors (egg allergy, peanut allergy or allergy to cow's milk proteins), airborne factors (acarids, pollen, animal dander) or contact factors (irritation by hard water or contact allergies to fragrances or to metals).

The treatment for this dermatosis is a symptomatic treatment which aims to control the inflammation and the pruritus so as to relieve the patient. The skin treatments should be daily, and therefore require good compliance by the patients: washing with a non-detergent product, application of a dermocorticoid to the eczema and of an emollient to the rest of the body. Thus, as for rosacea, the need continues for an effective treatment without any risk to the patient.

Psoriasis is a chronic dermatosis characterized by well-limited, often prurigenous, infiltrated erythematosquamous plaques (which itch). Pruritus is regularly present in patients who live in hot regions, but it is found only in 20 to 30% of patients in Northern Europe. The sites of predilection for psoriasis are the elbows, the knees, the thighs or the regions of friction or of microtrauma, and also the scalp. The pathogenesis of psoriasis is complex. The psoriatic lesion is characterized by epidermal hyperproliferation with an increase in keratinocytes and by moderate dermal and epidermal inflammations.

Psoriasis could be due to a genetic anomaly, associated with inflammatory processes. However, the signals involved in the dermal-epidermal interactions are not yet clearly understood.

The objective of anti-psoriatic treatments is to reduce the severity of the dermatosis to restore the patient's physical and psychological well-being. Current local treatments are used for the moderate forms of psoriasis, and systemic treatments are reserved for the severe forms. However, most anti-psoriatic treatments have a variable efficacy and more or less severe side effects, and are sometimes unpleasant to use. There exists therefore a need for an effective treatment without any risk to the patient.

### SUMMARY OF THE INVENTION

It has now surprisingly been discovered that the compound of formula (I) below (nepafenac) is useful for the treatment of dermatological conditions related to a keratinization disorder that may have an inflammatory immunoallergic component, whether regime or regimen, and more particularly very suitable for the treatment of rosacea, acne, psoriasis or atopic dermatitis (eczema):

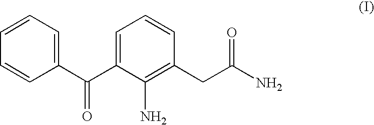

The compound of formula (I)—or 2-amino-3-benzoylphenylacetamide—known as nepafenac, is in particular described in U.S. Pat. Nos. 5,475,034 and 4,313,949. Such a compound has analgesic and antipyretic properties, and can be used for the treatment of ophthalmic pathologies. However, as is described in WO 02/13804, nepafenac can also be useful for the treatment of various retinopathies and cancers.

Thus, the present invention features the formulation of at least one compound of formula (I) or derivatives thereof:

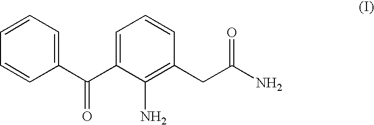

into pharmaceutical compositions useful for the treatment of dermatological conditions related to a keratinization disorder that may have an inflammatory immunoallergic component, advantageously rosacea, acne, psoriasis or atopic dermatitis (eczema).

The term “derivatives of the compound of formula (I)” means in particular the pharmaceutically acceptable salts, acids and hydrates.

The term “salts” means in particular the salts formed with a pharmaceutically acceptable acid or base. The salts of the compound of formula (I) are preferably the ammonium forms (—NH₃⁺) of this compound.

The term “acids” means preferably the carboxylic acid form of the compound of formula (I), i.e., in which the —NH₂ radical of the acetamide function is replaced with an —OH radical. Such a form corresponds to amfenac (2-amino-3-benzoylphenylacetic acid). The acid salts are also covered by the present invention; such salts are those formed from the acids of the compound of formula (I) and metal cations such as sodium, potassium, calcium, magnesium, zinc, copper or aluminum, preferably sodium.

The term “hydrates” means the compounds obtained by mixing with water.

The compound of formula (I) and derivatives thereof, and in particular nepafenac, can thus be formulated in pharmaceutical compositions. Said compositions comprise, in a pharmaceutically acceptable medium, at least one compound of formula (I) or derivatives thereof, preferably nepafenac.

The term “pharmaceutically acceptable medium” means a medium compatible with the skin, the mucous membranes and/or the superficial body growths.

### DETAILED DESCRIPTION OF BEST MODE AND SPECIFIC/PREFERRED EMBODIMENTS OF THE INVENTION

The compositions according to the invention advantageously comprise from 0.001% to 10% of compound of formula (I) or derivatives thereof, by weight, relative to the total weight of the composition. Preferably, the compositions according to the invention contain from 0.1% to 5% of compound of formula (I) or derivatives thereof, by weight, relative to the total weight of the composition.

The pharmaceutical compositions according to the invention are useful for the treatment of the skin and can be administered topically, parenterally or orally. Preferably, the composition is administered topically.

When administered orally, the pharmaceutical composition may be in liquid, pasty or solid form, in the form of powders, and more particularly in the form of tablets, gel capsules, sugar-coated tablets, syrups, suspensions, solutions, powders, granules, emulsions, or lipid or polymeric vesicles or nanospheres or microspheres for controlled release.

When administered parenterally, the composition may be in the form of solutions or suspensions for a drip or for injection.

Topical administration means a composition specifically suitable for application to the skin and not to the conjunctiva of the eye. Thus, the composition may be in liquid, pasty or solid form, and more particularly in the form of salves, creams, milks, ointments, powders, impregnated pads, syndets, wipes, solutions, gels, sprays, foams, suspensions, lotions, sticks, shampoos or washing bases. It may also be in the form of suspensions of lipid or polymeric vesicles or nanospheres or microspheres, or of polymeric patches and of hydrogels for controlled release. This composition for topical application may be in anhydrous form, in aqueous form or in the form of an emulsion.

In one preferred embodiment of the invention, the topical pharmaceutical composition is in the form of an emulsion of cream or lotion type, of a gel or of a solution.

When the composition according to the invention is in the form of an emulsion, it comprises at least one surfactant. In fact, the conventional emulsions as described in the prior art are virtually homogeneous, unstable systems of two immiscible liquids, one of which is dispersed in the other in the form of fine droplets (micelles). This dispersion is stabilized by virtue of the action of surfactant emulsifiers which modify the structure and the ratio of the forces at the level of the interface, and therefore increase the stability of the dispersion by reducing the interfacial tension energy.

Surfactant emulsifiers are amphiphilic compounds which possess a hydrophobic part having an affinity for oil and a hydrophilic part having an affinity for water, thus creating a link from the two phases. Ionic or non-ionic emulsifiers therefore stabilize oil/water emulsions by adsorbing at the interface and forming lamellar layers of liquid crystals.

The emulsifying capacity of non-ionic surfactants is closely linked to the polarity of the molecule. This polarity is defined by the HLB (hydrophilic/lipophilic balance). Conventional emulsions are generally stabilized with a mixture of surfactants, the HLBs of which can be quite different, but the proportion of which in the mixture corresponds to the required HLB of the fatty phase to be emulsified.

Among the surfactants that can be used according to the invention, exemplary are, by way of examples, of the glyceryl/PEG100 stearate marketed under the trademark Arlacel 165FL by Uniqema or under the trademark Simulsol 165 by SEPPIC, polyoxyethylenated fatty acid esters, such as Arlatone 983 from the company Uniqema or the polyoxyethylenated (2) stearyl alcohol marketed under the trademark Brij72 combined with the polyethylenated (21) stearyl alcohol marketed under the trademark Brij721 by Uniqema, sorbitan esters such as the sorbitan oleate marketed under the trademark Arlacel 80 by ICI or marketed under the trademark Crill 4 by Croda, the sorbitan sesquioleate marketed under the trademark Arlacel 83 by ICI or marketed under the trademark Montane 83 by Seppic, or else sorbitan isostearate; fatty alcohol ethers.

The compositions according to the invention advantageously comprise up to 15% by weight of suitable surfactant emulsifier, preferably from 2% to 12% by weight, and more particularly from 2% to 6% by weight, relative to the total weight of the composition.

The composition in emulsion form thus comprises:

a) an oily phase comprising fatty substances;

b) at least one surfactant emulsifier;

c) at least one compound selected from among the compound of formula (I) and derivatives thereof;

d) one or more solvents and/or propenetrating agents for the active agent(s); and

e) water.

The oily phase of the composition according to the invention may comprise, for example, plant, mineral, animal or synthetic oils, silicone oils, Guerbet alcohols or other fatty substances, and mixtures thereof.

Examples of a mineral oil include paraffin oils of various viscosities, such as Primol 352, Marcol 82 or Marcol 152, marketed by Esso.

As a plant oil, exemplary are sweet almond oil, palm oil, soy oil, sesame oil or sunflower oil.

As an animal oil, exemplary are lanolin, squalene, fish oil or mink oil.

As a synthetic oil, exemplary are esters, such as the cetearyl isononanoate marketed in particular under the trademark Cetiol SN by Cognis France, diisopropyl adipate, such as the product marketed under the trademark Ceraphyl 230 by ISF, isopropyl palmitate, such as the product marketed under the trademark Crodamol IPP by Croda, or caprylic/capric triglyceride, such as Miglyol 812 marketed by Huls/Lambert Rivière.

As a silicone oil, exemplary are a dimethicone, such as the product marketed under the trademark Dow Corning 200 fluid, a cyclomethicone, such as the product marketed under the trademark Dow Corning 244 fluid by Dow Corning, or the product marketed under the trademark Mirasil CM5 by SACI-CFPA.

As other fatty substances, exemplary are fatty acids such as stearic acid, fatty alcohols such as stearyl alcohol, cetostearyl alcohol and cetyl alcohol, or derivatives thereof, waxes such as beeswax, carnauba wax or candelilla wax, and also gums, in particular silicone gums.

The ingredients of the oily phase may be selected in a varied manner by those skilled in the art, to prepare a composition having the desired properties, for example in terms of consistency or in terms of texture.

Preferably, the oily phase of the composition according to the invention comprises a synthetic oil and/or a silicone oil; as synthetic oil, isopropyl palmitate, such as the product marketed under the trademark Crodamol IPP by Croda, or isopropyl myristate, such as the product marketed under the trademark Crodamol IPM by Croda, is preferred, and as silicone oil, a dimethicone is preferred.

The oily phase of the emulsion according to the invention may be present at a content of from 3% to 50% by weight, relative to the total weight of the composition, and preferably from 6% to 20% by weight.

By way of example of a solvent and/or a propenetrating agent for the compound of formula (I) or derivatives thereof, mention will preferentially be made of propylene glycol, alcohols such as ethanol, isopropanol or butanol, N-methyl-2-pyrrolidone or DMSO, polysorbate 80, phenoxyethanol, and mixtures thereof.

The composition of the invention contains from 0.1% to 20%, and preferentially from 1% to 10%, of a solvent and/or propenetrating agent for the compound of formula (I) or derivatives thereof.

The compositions of the invention also contain water ranging from 30% to 95%, and preferentially from 60% to 80%, by weight, relative to the total weight of the composition. The water used in the composition according to the invention will preferably be purified water.

The compositions according to the invention may also be in the form of a gel; these then comprise one or more gelling compounds, ranging from 0.01% to 5% by weight, relative to the total weight of the composition. Among the gelling agents that can be used in the composition according to the invention, exemplary are carboxyvinyl polymers (carbomers), and, by way of non-limiting examples of a carbomer, exemplary are Carbopol 981, Carbopol ETD 2020, Carbopol 980, Carbapol Ultrez 10 NF or Pemulen TR1, marketed by Noveon.

Also exemplary are cellulosic derivatives, for instance hydroxypropylmethylcellulose or hydroxyethylcellulose; xanthan gums, aluminum/magnesium silicates, such as Veegum K or Veegum Ultra marketed by Vanderbilt, guar gums and the like, polyacrylamides such as the polyacrylamide/isoparaffin C13-14/laureth-7 mixture, for instance that marketed by Seppic under the trademark Sepigel 305 or the acrylamide/AMPS copolymer dispersion 40%/isohexadecane mixture under the trademark Simulgel 600 PHA, or the family of modified starches, such as Structure Solanace marketed by National Starch, or mixtures thereof.

The compositions of the invention preferentially contain from 0.01% to 5%, and preferably from 0.1% to 3%, of gelling agent.

When the composition is in the form of a solution, it comprises, in addition to the compound of formula (I) or derivatives thereof, an aqueous or oily solution and, optionally, one or more solvents and/or propenetrating agents for the active agents as described above.

The pharmaceutical composition according to the invention may also contain inert additives or combinations of these additives, such as:

preservatives;

stabilizers;

emollients;

moisture regulators;

pH regulators;

osmotic pressure modifiers;

UV-A and UV-B screens;

antioxidants.

Of course, one skilled in the art will take care to select the optional compound(s) to be added to these compositions in such a way that the advantageous properties intrinsically associated with the present invention are not, or are not substantially, impaired by the addition envisaged.

These additives may be present in the composition at from 0.001% to 20% by weight, relative to the total weight of the composition.

The administration of the compound of formula (I) or derivatives thereof, as a medicament, and more particularly as a topical pharmaceutical composition, is particularly for the treatment of rosacea, of psoriasis or of atopic dermatitis (eczema).

To further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Composition 1

| | % by weight relative to the total |
|---|---|
| Ingredients | weight of the composition |
| Nepafenac | 1.00 |
| EDTA | 0.1 |
| Polysorbate 80 | 8.0 |
| Propylene glycol | 20.00 |
| Benzyl alcohol | 3 |
| Water | Qs 100 |

### Example 2

### Composition 2

| | % by weight relative to the total |
|---|---|
| Ingredients | weight of the composition |
| Amfenac | 1.00 |
| Glycerol | 4.0 |
| Steareth-2 | 1.0 |
| Steareth-21 | 2.0 |
| Aluminum magnesium silicate/titanium | 1.0 |
| dioxide/silica | |
| Methyl para-hydroxybenzoate | 0.2 |
| Propyl para-hydroxybenzoate | 0.1 |
| Disodium EDTA | 0.05 |
| Citric acid monohydrate | 0.05 |
| Isopropyl palmitate | 4.0 |
| Glyceryl/PEG 100 stearate | 2.0 |
| Self-emulsifiable wax | 1.0 |
| Palmitostearic acid | 2.00 |
| Dimethicone 200-350 cS | 0.5 |
| Propylene glycol | 4.0 |
| Glyceryl triacetate | 1.00 |
| Phenoxyethanol | 0.5 |
| 10% sodium hydroxide | Qs pH |
| Water | Qs 100 |

### Example 3

### Biological Test

The evaluation of a compound of formula (I) is carried out in a model of inflammation induced by the topical application of a solution of arachidonic acid to the mouse ear. The intensity of the inflammatory response is subsequently evaluated by measuring the thickness of the ear at 1, 2 and 6 h, which reflects the oedematous response. The activity of the compound of formula (I) is characterized by the percentage inhibition of the response compared with the untreated animals.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment of a dermatological/keratinization disorder that may have an inflammatory immunoallergic component, comprising administering to a subject in need of such treatment, a thus effective amount of a pharmaceutical composition which comprises at least one compound having the formula (I) or derivative thereof: 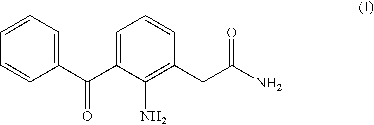
formulated into a pharmaceutically acceptable medium therefor.

2. A regime or regimen for the treatment of rosacea, acne, psoriasis or atopic dermatitis, comprising administering to a subject in need of such treatment, a thus effective amount of a pharmaceutical composition which comprises at least one compound having the formula (I) or derivative thereof: 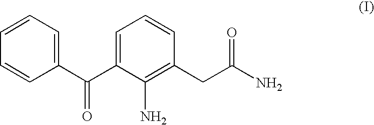
formulated into a pharmaceutically acceptable medium therefor.

3. The regime or regimen as defined by claim 1, said pharmaceutical composition comprising at least one salt, acid and/or hydrate of said compound having the formula (I).

4. The regime or regimen as defined by claim 1, said pharmaceutical composition comprising nepafenac or amfenac.

5. The regime or regimen as defined by claim 1, said pharmaceutical composition being in a form suited for oral administration.

6. The regime or regimen as defined by claim 1, said pharmaceutical composition being in a form suited for topical administration.

7. The regime or regimen as defined by claim 1, said pharmaceutical composition being in a form suited for parenteral administration.

8. The regime or regimen as defined by claim 6, said pharmaceutical composition comprising a cream or lotion emulsion, a gel or a solution.

9. The regime or regimen as defined by claim 1, said pharmaceutical composition comprising 0.001% to 10% by weight of said at least one compound of formula (I) or derivatives thereof.

10. The regime or regimen as defined by claim 1, said pharmaceutical composition comprising 0.1% to 5% by weight of said at least one compound of formula (I) or derivatives thereof.

11. The regime or regimen as defined by claim 2, comprising the treatment of rosacea.

12. The regime or regimen as defined by claim 2, comprising the treatment of acne.
